(19) United States
(12) Patent Application Publication
(10) Pub. No.: US 2026/0294377 A1
(43) Pub. Date: Oct. 1, 2026

(54) CUMULATIVE PATIENT X-RAY RADIATION DOSE PROFILE BASED ON SCOUT IMAGES INDEPENDENT OF Z-AXIS REFERENCE ORIGINS

(71) Applicant: GE Precision Healthcare LLC, Waukesha, WI (US)

(72) Inventors: Alexandre Antoine Pierre Gallo, Strasbourg (FR); Célia Tiffany Laurène Wengert, Strasbourg (FR)

(73) Assignee: GE Precision Healthcare LLC, Waukesha, WI (US)

(21) Appl. No.: 19/097,332

(22) Filed: Apr. 1, 2025

(51) Int. Cl. A61B 6/00; A61B 6/03; A61B 6/58

(52) U.S. Cl. CPC A61B 6/542; A61B 6/032; A61B 6/583

(57) ABSTRACT

A computer-implemented method includes receiving at least a first scout image with a first dose profile and a second scout image with a second dose profile for a patient. The first scout image includes a first reference origin, and the second scout image includes a second reference origin. The method further includes receiving a reference image that is pre-marked with a set of anatomical regions. The method further includes mapping anatomical regions between the first scout image and the reference image and the second scout image and the reference image. The method further includes summing the first dose profile and the second dose profile based on the mapped anatomical regions between the first and second scout images and the reference image, independent of the first and second reference origins. The method further includes generating a cumulative dose profile for the patient based on the summed first and second dose profiles.

### FIELD

The following generally relates to medical imaging, and more particularly to a cumulative patient X-ray radiation dose profile that is based on scout images independent of Z-axis reference origins.

### BACKGROUND

A computed tomography (CT) scanner includes a gantry and a rotating frame rotatably supported by a bearing in the gantry. The rotating frame is configured to rotate around a bore along an axis of rotation (Z-axis) about a center of rotation (i.e., an isocenter). The rotating frame carries components such as an X-ray source, an X-ray radiation sensitive detector array, a Data Acquisition System (DAS), etc. For axial and/or helical scans, the rotating frame rotates around the bore, the X-ray source emits X-ray radiation that traverses the isocenter (and a patient supported by a cradle in the bore) and is detected by the X-ray radiation sensitive detector array. The patient is positioned at a particular Z-axis location for each axial scan and moves through the bore, via the cradle, for a helical scan.

The DAS generates and outputs projection data (line integrals) indicative of the sensed X-ray radiation. A reconstructor reconstructs the projection data and generates three-dimensional (3-D) volumetric image data. Voxels of the reconstructed volumetric image data are displayed as a two-dimensional (2-D) image/slice and/or a three-dimensional (3-D) rendering, both at least using gray scale values corresponding to a relative radiodensity. The gray scale values reflect the attenuation characteristics of the scanned patient and generally show structure such as anatomical structures within the patient. A user can scroll through slices, rotate, zoom and/or pan images, adjust window/level settings, adjust slice thickness, take measurements, add color overlays, etc.

A CT imaging examination includes one or more series of groups, each including one or more volume scans. In general, groups capture different phases, such as pre-contrast or post-contrast, different views such as different anatomical planes (e.g., axial, coronal, sagittal, oblique, etc.), and/or other information, and volume scans within a group capture anatomy at different positions along a length of the body of the patient. An example includes a series with a first volume scan of the thorax (which may include part of the neck, the shoulders, the chest, the abdomen, the pelvis, part of the legs, etc.) and a second volume scan of the head (which may also include the part of the neck and part of shoulders).

One or more scout (also referred to as a localizer, a scanogram, a topogram, a surview, a pilot, a pre-scan, etc.) images are first acquired and utilized to assist with planning the volume scans of the series. Generally, a scout image is a 2-D projection image along a portion of a longitudinal axis of a patient, similar to an X-ray radiograph. It is acquired by translating a patient, via the cradle, through the bore while the X-ray source emits X-ray radiation from a static angular position. For example, where the patient is in a supine position, the X-ray source has been positioned at 0° azimuth at a front of the patient for an anteroposterior (A/P) scout image. In another example, again where the patient is in a supine position, the X-ray source has been positioned at 90° azimuth at a side of the patient for a lateral (Lat) scout image.

Scout image acquisitions generally are low-dose, low resolution acquisitions since scout images are utilized to provide a basic overview of a section of the body that includes the region to be imaged for planning a volume scan and not for diagnostic imaging. The planning includes utilizing the one or more scout images to identify a start scan position and an end scan position (or a scan extent), to position the patient, etc. for a volume scan. Planning further includes setting parameters such as kVp, mAs, etc. A denser region requires a higher X-ray radiation dose (i.e., the amount of energy deposited in tissues during an scan) than a less dense region to generate diagnostic quality volumetric image data. For example, a scan of the head would require a higher X-ray radiation dose relative to a scan of the lungs.

A higher X-ray radiation dose results in clearer and more detailed images with a contrast that allows for distinguishing between normal and abnormal tissues and reduced image artifact that could otherwise visually obscure details, all of which contribute to reconstructing diagnostic quality images. However, X-ray radiation is ionizing radiation, which can damage and/or kill cells, and potentially increase a likelihood of cancer. Computed Tomography Dose Index Volume (CTDIᵥₒₗ) is a standardized metric in CT imaging to estimate the X-ray radiation dose delivered to a patient during a CT scan. CTDIᵥₒₗ values provide a weighted average dose of X-ray radiation absorbed within a specific slice or volume of tissue. Size Specific Dose Estimate (SSDE) is another standardized metric in CT imaging to estimate the X-ray radiation dose delivered to a patient during a CT scan. SSDE, in general, corrects the CTDIᵥₒₗ by taking into account a diameter of the patient. Image quality and X-ray radiation dose are considered when planning a volume scan to provide a lowest possible X-ray radiation dose, while reconstructing diagnostic quality volumetric image data.

Again, one or more scout images are acquired for each imaging examination of a patient. However, all of the scout images for a particular imaging examination may not have the same frame of reference. For example, the Z-axis reference origin between scout images in a same imaging examination may be reset between scout image acquisitions such that they have different Z-axis origins. For instance, the Z-axis origin for a pelvic scan may be around the leg region, whereas the Z-axis origin for a head scan may be around the neck or shoulder region. The Z-axis reference origin for different imaging examinations will necessarily be different. A cumulative X-ray radiation dose has been determined for scout images that share a same Z-axis reference origin. However, there currently is no effective approach for determining a cumulative X-ray radiation dose for scout images with different Z-axis reference origins. As a consequence, a patient may be exposed to an unnecessary and/or untracked X-ray radiation dose, including overlapping irradiated regions across volume scans.

In view of at least the foregoing, there is an unresolved need for another approach for determining a cumulative X-ray radiation dose profile that at least mitigates shortcoming of existing approaches.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a computer-implemented method includes receiving at least a first scout image with a first dose profile and a second scout image with a second dose profile for a patient. The first scout image includes a first reference origin, and the second scout image includes a second reference origin. The computer-implemented method further includes receiving a human reference image that is pre-marked with a set of anatomical regions. The computer-implemented method further includes mapping anatomical regions between the first scout image and the human reference image and the second scout image and the human reference image. The computer-implemented method further includes summing the first dose profile and the second dose profile based on the mapped anatomical regions between the first and second scout images and the human reference image, independent of the first and second reference origins. The computer-implemented method further includes generating a cumulative dose profile for the patient based on the summed first and second dose profiles.

In another aspect, a computer readable medium is encoded with computer executable instructions. The computer executable instructions, when executed by a processor, cause the processor to receive at least a first scout image with a first dose profile and a second scout image with a second dose profile for a patient. The first scout image includes a first reference origin, the second scout image includes a second reference origin. The instructions further cause the processor to receive a human reference image that is pre-marked with a set of anatomical regions. The instructions further cause the processor to map anatomical regions between the first scout image and the human reference image and the second scout image and the human reference image. The instructions further cause the processor to sum the first dose profile, and the second dose profile based on the mapped anatomical regions between the first and second scout images and the human reference image, independent of the first and second reference origins. The instructions further cause the processor to generate a cumulative dose profile for the patient based on the summed first and second dose profiles.

In another aspect, a system includes a memory and at least one processor. The memory includes a cumulative X-ray radiation dose generating module. The at least one processor is configured to execute instructions of the cumulative X-ray radiation dose generating module. The instructions cause the processor to receive at least a first scout image with a first dose profile and a second scout image with a second dose profile for a patient. The first scout image includes a first reference origin, and the second scout image includes a second reference origin. The instructions further cause the processor to receive a human reference image that is pre-marked with a set of anatomical regions. The instructions further cause the processor to map anatomical regions between the first scout image and the human reference image and the second scout image and the human reference image. The instructions further cause the processor to sum the first dose profile, and the second dose profile based on the mapped anatomical regions between the first and second scout images and the human reference image, independent of the first and second reference origins. The instructions further cause the processor to generate a cumulative dose profile for the patient based on the summed first and second dose profiles.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example of an imaging system configured for computed tomography (CT) imaging and including at least application software, an X-ray radiation dose determining module, and a cumulative X-ray radiation dose profile generating module, in accordance with an embodiment(s) herein.

FIG. 2 schematically illustrates a non-limiting example of a system configured for computed tomography (CT) imaging and including the application software, the X-ray radiation dose determining module, and the cumulative X-ray radiation dose profile generating module, in accordance with an embodiment(s) herein.

FIG. 3 schematically illustrates a non-limiting example of the cumulative X-ray radiation dose profile generating module, in accordance with an embodiment(s) herein.

FIG. 4 schematically illustrates a non-limiting example of a first scout body image of a patient including part of a head region and a neck and shoulders region through an legs region, in accordance with an embodiment(s) herein.

FIG. 5 schematically illustrates a non-limiting example of a second scout image of a head of the patient including the head region and part of the neck and shoulders region, in accordance with an embodiment(s) herein.

FIG. 6 schematically illustrates a non-limiting example of the first scout image delineated with a predetermined set of anatomical regions, including a head region, a neck and shoulders region, an abdomen region, a pelvic region, and a legs region, in accordance with an embodiment(s) herein.

FIG. 7 schematically illustrates a non-limiting example of the second scout image delineated with a predetermined set of anatomical regions, including a head region and a neck and shoulders region, in accordance with an embodiment(s) herein.

FIG. 8 schematically illustrates a non-limiting example of a human reference image delineated with a predetermined set of anatomical regions, including a head region, a neck and shoulders region, an abdomen region, a pelvic region, and a leg region, in accordance with an embodiment(s) herein.

FIG. 9 schematically illustrates a non-limiting example of mappings of delineated anatomical regions between the first scout image and the human reference image and between the second scout image and the human reference image, in accordance with an embodiment(s) herein.

FIG. 10 schematically illustrates a non-limiting example of determining a cumulative X-ray radiation dose profile across scout images with overlapping anatomical regions, in accordance with an embodiment(s) herein.

FIG. 11 schematically illustrates a non-limiting example of the cumulative X-ray radiation dose profile generating module, in accordance with an embodiment(s) herein.

FIG. 12 schematically illustrates a non-limiting example of a first mask image generated from the first scout image, in accordance with an embodiment(s) herein.

FIG. 13 schematically illustrates a non-limiting example of a second mask image generated from the second scout image, in accordance with an embodiment(s) herein.

FIG. 14 schematically illustrates a non-limiting example of adding shoulder markers on the first mask image for a scout image with the arms raised above the head of a patient, in accordance with an embodiment(s) herein.

FIG. 15 schematically illustrates a non-limiting example of placing shoulder markers on the first mask image for a scout image with the arms at sides of the patient, in accordance with an embodiment(s) herein.

FIG. 16 schematically illustrates a non-limiting example of placing a crotch marker on the first mask image, in accordance with an embodiment(s) herein.

FIG. 17 schematically illustrates a non-limiting example of placing a top of the head marker on the second mask image, in accordance with an embodiment(s) herein.

FIG. 18 schematically illustrates a non-limiting example of placing shoulder markers on the second mask image, in accordance with an embodiment(s) herein.

FIG. 19 schematically illustrates a non-limiting example of a human reference image with a top of the head marker, shoulder markers and crotch marker, along with a predetermined set of anatomical regions, including a head region, a neck and shoulders region, an abdomen region, a pelvic region, a legs region, and a legs region, in accordance with an embodiment(s) herein.

FIG. 20 schematically illustrates a non-limiting example of aligning the markers of the first mask image and the second mask image with the corresponding markers of the human reference image, in accordance with an embodiment(s) herein.

FIG. 21 schematically illustrates a non-limiting example of combining the first scout image with the anatomical regions, the second scout image with the anatomical regions, and the reference image with the anatomical regions, in accordance with an embodiment(s) herein.

FIG. 22 illustrates a non-limiting example of a flow chart for a method of determining a cumulative patient X-ray radiation dose profile that is based on scout images with different reference origin, in accordance with an embodiment(s) herein.

FIG. 23 illustrates another non-limiting example of a flow chart for a method of determining a cumulative patient X-ray radiation dose profile that is based on scout images with a different reference origin, in accordance with an embodiment(s) herein.

FIG. 24 illustrates yet another non-limiting example of a flow chart for a method of determining a cumulative patient X-ray radiation dose profile that is based on scout images with a different reference origin, in accordance with an embodiment(s) herein.

FIG. 25 schematically illustrates a non-limiting example of determining a cumulative X-ray radiation dose profile across scout images with continuous anatomical regions, in accordance with an embodiment(s) herein.

FIG. 26 schematically illustrates a non-limiting example of determining a cumulative X-ray radiation dose profile across scout images with a gap between anatomical regions, in accordance with an embodiment(s) herein.

### DETAILED DESCRIPTION

Embodiments of the present disclosure will now be described, by way of example, with reference to the figures, in which a system, a method and/or instructions of a computer readable medium determine a cumulative X-ray radiation dose profile for scout images independent of Z-axis reference origins to generate a cumulative X-ray radiation dose map for a patient over time. The scout images may correspond to a same and/or different imaging examination, have a same and/or different Z-axis reference origin, and/or include overlapping regions, contiguous or non-overlapping and non-contiguous anatomical regions. In one instance, the cumulative X-ray radiation dose map is utilized for evaluating X-ray radiation dose for a patient over time, allowing clinicians to follow the anatomical areas irradiated by each acquisition, and/or improve X-ray radiation dose monitoring and/or management of patients'radiological exposures and/or a precision of exposure protocols.

As discussed above, one or more scout images are acquired for each imaging examination of a patient, and not all of the scout images for a particular imaging examination and/or all of the scout images across imaging examinations for the patient will have a same Z-axis reference origin, e.g., the reference origin may be reset between volumes scans and is reset between imaging examinations. In addition, there currently is no effective approach for determining a cumulative X-ray radiation dose profile over time for scout images of a patient that have different Z-axis reference origins. As a consequence, a patient may be exposed to an unnecessary X-ray radiation dose and/or untracked X-ray radiation dose corresponding to overlapping irradiated regions. The approach described herein determines a cumulative X-ray radiation dose profile over time for scout images, including scout images with different Z-axis reference origins.

As described in greater detail below, this is achieved by determining a cumulative X-ray radiation dose profile over time based on a set of predetermined anatomical regions, independent of the Z-axis reference origins of the scout images. In one instance, each scout image is delineated into a plurality of anatomical regions, the anatomical regions are mapped to pre-marked anatomical regions of a human reference image, and the X-ray radiation dose of anatomical regions of each scout image is projected to corresponding anatomical regions of the human reference image. In another instance, a mask image is generated for each scout image, a set of anatomical markers are added to the mask images, the mask images are aligned with a human reference image based on the anatomical markers, anatomical region of the reference image are mapped to the aligned mask images and then mapped therefrom to the scout images, and the scout images and human reference image are combined, which maps the X-ray radiation dose of each scout image to the human reference image. As utilized herein, a human reference image encompasses an image with a human shape that can be anatomically mapped to one or more anatomical regions in a scout, mask, and/or other image, such as a graphical anatomical phantom, model, atlas, profile, silhouette, surface, perimeter, etc. In both instances, the X-ray radiation dose of the scout images is summed to generate a cumulative X-ray radiation dose profile.

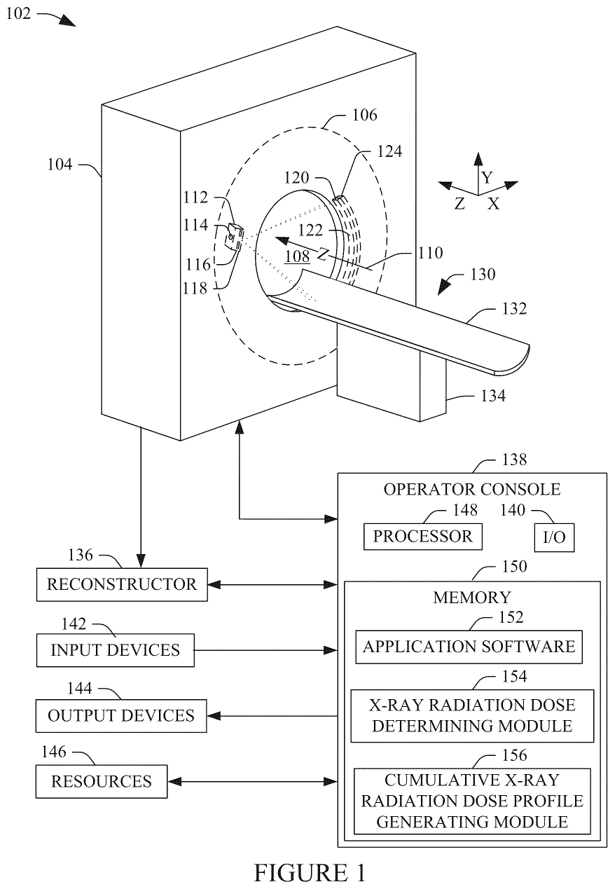

Initially referring to FIG. 1, a non-limiting example of an imaging system 102 such as a computed tomography (CT) imaging system is schematically illustrated. The imaging system 102 includes a gantry 104. In some instances, the gantry 104 is configured to tilt. The imaging system 102 further includes a rotating frame 106. The rotating frame 106 is rotatably supported in the gantry 104, e.g., via a bearing (e.g., a slip ring) or the like, and is configured to rotate around a bore 108 about a rotational or z-axis 110, which extends through a center of rotation/a center of the bore 108 (i.e., an isocenter). A gantry controller (not visible) is configured to control rotation of the rotating frame 106 and, if configured to tilt, tilting of the gantry 104.

An X-ray source assembly 112 is supported by the rotating frame 106 and rotates in coordination with the rotating frame 106. The X-ray source assembly 112 includes an X-ray source 114 such as an X-ray tube. The X-ray source 114 is configured to emit X-ray radiation having an energy in the X-ray diagnostic range (e.g., 20 keV to 150 keV). The X-ray source assembly 112 may further include or is coupled to a filter 116 that characterizes an X-ray radiation dose profile and/or a collimator 118 that shapes the X-ray radiation to form a generally fan, wedge, cone, etc. shaped beam that traverses the examination region 108. An X-ray controller (not visible) is configured to control components of the X-ray assembly 112 such as X-ray radiation emission of the X-ray source 114, the collimator 118, etc.

An X-ray radiation sensitive detector array 120 includes a one-dimensional (1-D) or two-dimensional (2-D) array of rows of X-ray radiation sensitive detector elements 122 and is supported by the rotating frame 106 along an arc opposite the X-ray source 114, across the bore 108. Each of the X-ray radiation sensitive detector elements 122 is in electrical communication with a data acquisition system (DAS) 124. A DAS controller (not visible) controls the X-ray radiation sensitive detector array 120.

A table 130 includes a cradle 132 moveably coupled to a frame/base 134. In one instance, the cradle 132 is slidably coupled to the frame/base 134 via a bearing or the like, and a drive system (not visible) including a controller, a motor, a lead screw, and a nut (or other drive system) translates the cradle 132 along the frame/base 134 into and out of the examination region 108. The cradle 132 is configured to support an object or subject in the examination region 108 for loading, scanning, and/or unloading the subject or object. A table controller (not visible) controls the drive system.

For a scout acquisition, the X-ray source 114 is positioned at a static angular position (e.g., 0°, 90°, etc.) and emits X-ray radiation while the cradle 132 moves the patient through the bore 108. For axial acquisitions, the X-ray source 114 rotates around the bore 108 and emits X-ray radiation while the cradle 132 is positioned at a statis Z-axis position for each integration period and moves between integration periods. For a helical acquisition, the X-ray source 114 rotates around the bore 108 and emits X-ray radiation while the cradle 132 moves the patient through the bore 108 over consecutive arc segments (integration periods) each revolution, and generate respective signals. For each integration period, projection data is generated.

A reconstructor 136 reconstructs the projection data and generates a two-dimensional (2-D) scout image for each scout acquisition, individual axial two-dimensional (2-D) images for each step and shoot acquisition and/or volumetric three-dimensional (3-D) image data for each helical acquisition. The scout image, the individual axial images and/or the 3-D volumetric image data (and/or 2-D slices thereof) can be visually presented, filmed, etc. Examples of suitable reconstruction algorithms include filtered back projection (FBP), advanced statistical iterative reconstruction (ASIR), conjugate gradient (CG), maximum likelihood expectation maximization (MLEM), model-based iterative reconstruction (MBIR), and/or other reconstruction algorithm.

A computing system 138 serves as an operator console of the imaging system 102 and includes a computer, a workstation, a server, etc. The computing system 138 includes input/output (I/O) 140. One or more input devices 142 include a keyboard, mouse, touchscreen, microphone, etc. One or more output devices 144 include a human readable device such as a display monitor or the like. One or more resources 146 include one or more of a server, a workstation, a Radiology Information System (RIS), a Hospital Information System (HIS), an Electronic Medical Record (EMR), a Picture Archiving and Communications System (PACS), another scanner, cloud processing resources, etc. The one or more input device 142, the one or more output device 144 and/or the one or more remote resource 146 are in electrical communication with the computing system 138 via the I/O 140 and/or otherwise.

The computing system 138 further includes at least one processor 148 such as a microprocessor (mP), a central processing unit (CPU), graphics processing unit (GPU), etc., and a computer readable medium 150 (“MEMORY”), which includes non-transitory medium and excludes transitory medium (signals, carrier waves, and the like). The at least one processor 148 is configured to execute computer readable instructions included in the computer readable medium 150. In this example, the computer readable medium 150 includes at least application software 152, an X-ray radiation dose determining module 154, and a cumulative X-ray radiation dose profile generating module 156.

The application software 152 provides a user interface, such as a graphical user interface (GUI), that allows user to set up an imaging examination (e.g., a scout acquisition, start and end scan positions, kVp, mAs, etc. for a volume scan), start a scan, view images, transfer images, etc., utilizing an input device such as a keyboard, a mouse, etc. from the one or more input devices 142 and/or an output device such as a display monitor of the one or more output devices 144. Again, a patient may have one or more CT imaging examinations, each CT imaging examination may include one or more series of groups, and each series may include one or more scout images and one or more volume scans, where the one or more scout images are utilized to plan the one or more volume scans.

The X-ray radiation dose determining module 154 is configured to at least determine a X-ray radiation dose (e.g., a CTDIᵥₒₗ, an SSDE, etc.) for each scout image of an imaging examination via known and/or other approaches. In one instance, the X-ray radiation dose for a scout image is stored in metadata of the scout image and can be accessed through the operator console 138 and/or included in a DICOM header and/or field of a file transferred to a PACS and/or other system of the one or more resources 146. As discussed above, one or more scout images are acquired for each imaging examination of a patient, and not all of the scout images for a particular imaging examination and/or across imaging examination for the patient will have a same Z-axis reference origin, and there currently is no effective approach for aggregating the X-ray radiation dose values across such scout images. As a consequence, a patient may be exposed to unnecessary and/or untracked X-ray radiation dose corresponding to overlapping irradiated regions.

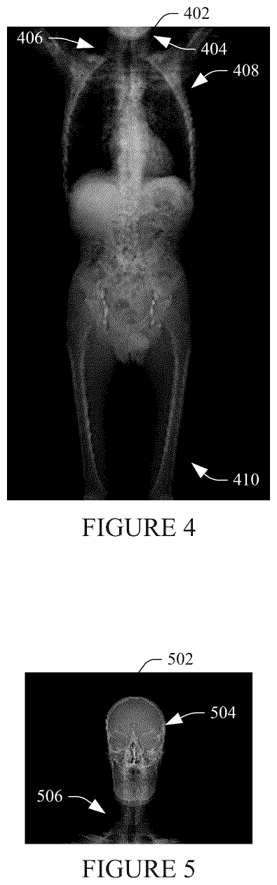

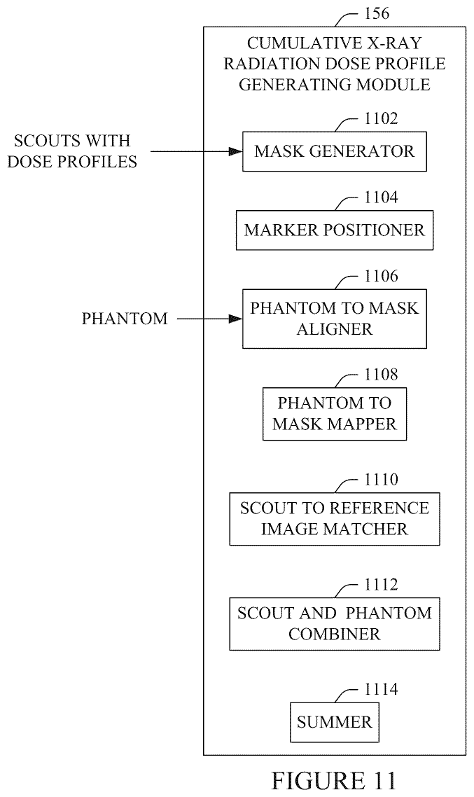

The cumulative X-ray radiation dose profile generator module 156 is configured to sum X-ray radiation dose across scout images with different Z-axis reference origins to determine a cumulative X-ray radiation dose profile, based on a set of predetermined anatomical regions, independent of the Z-axis reference origins of the scout images. As described in greater detail below, in one instance (e.g., FIGS. 4-10, 22 and 23) each scout image is delineated into a plurality of anatomical regions, the anatomical regions are mapped to pre-marked anatomical regions of a human reference image, and the X-ray radiation dose of anatomical regions of each scout image is projected to corresponding anatomical regions of the reference image. In another instance (e.g., FIGS. 11-22 and 24), a mask image is generated for each scout image, a set of anatomical markers are added to the mask images, the mask images are aligned with a human reference image based on the anatomical markers, anatomical region of the reference image are mapped to the aligned mask images and then mapped therefrom to the scout images, and the scout images and human reference image are combined, which maps the X-ray radiation dose of each scout image to the human reference image.

In either, both and/or other instances, the X-ray radiation dose of the scout images is summed based on the anatomical regions of the human reference image, generating an X-ray radiation dose profile with an X-ray radiation dose map over time. The cumulative X-ray radiation dose profile can be stored locally in the imaging system 102. Additionally, or alternatively, the cumulative X-ray radiation dose profile generator module 156 can transmit the cumulative X-ray radiation dose profile to one or more resources of the one or more resources 146, e.g., in an EMR for the patient, a HIS, an RIS, a PACS, other computing system, etc. Additionally, or alternatively, the cumulative X-ray radiation dose profile generator module 156 can display the cumulative X-ray radiation dose profile in a display monitor or the like of the one or more output devices 144 for visual and/or other observation.

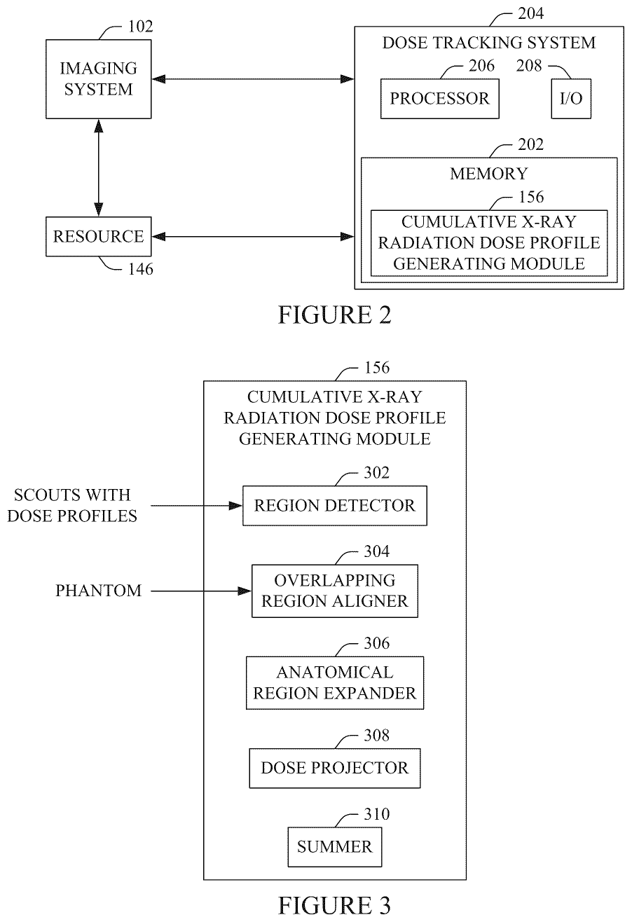

In FIG. 1, the cumulative X-ray radiation dose profile generator module 156 is included in the memory 150 of the operator console 138 of the imaging system 102. Additionally, or alternatively, the cumulative X-ray radiation dose profile generator module 156 is included in a memory of one or more other computing systems. FIG. 2 schematically illustrates an example in which the cumulative X-ray radiation dose profile generator module 156 is included in the memory 202 of a computing system 204 that also include a processor 206 and I/O 208, which are similar to the processor 148 and I/O 140 described in connection with FIG. 1 and thus are not described in detail here. In this example, the computing system 204 is a dedicated dose tracking system. In other instance, the computing system 204 is not a dedicated dose tracking system.

The memory 202, the processor 206 and the I/O 208 of the computing system 204 are similar to the memory 150, the processor 148 and the I/O 140 of the imaging system 102 described in connection with FIG. 1 and thus are not described in detail here, e.g., for sake of brevity and/or clarity. In another example, the computing system 204 further includes the X-ray radiation dose determining module 154. In another example, the computing system 204 includes a resource of the one or more resources 146 described in connection with FIG. 1 such as a HIS, an RIS, a PACS, a cloud-based resource, and/or other computing system.

Turning to FIG. 3, an example of the cumulative X-ray radiation dose profile generating module 156 is schematically illustrated. This example is discussed in connection with FIGS. 4, 5, 6, 7, 8, 9 and 10. Another example of the cumulative X-ray radiation dose profile generating module 156 is described in connection with FIGS. 11, 12, 13, 14, 15, 16, 17, 18, 19, 20 and 21. Other examples are also contemplated herein.

Beginning with FIG. 3, the cumulative X-ray radiation dose profile generating module 156 receives, as input, multiple scout images (some having different Z-axis reference origins) along with individual X-ray radiation dose profiles for each of the scout images. FIG. 4 graphically depicts an example of a first scout image 402 of a patient. The first scout image 402 includes a head region 404 and a neck and shoulders region 406, as well at other regions 408 through a legs region 410 of the patient. FIG. 5 graphically depicts an example of a second scout image 502 of the patient. The second scout image 502 includes a head region 504 and a neck and shoulders region 506. The head regions 404 and 504 both include the head of the patient, in its entirety or in part, and the neck and shoulders region 406 and 506 includes the neck and shoulders of the patient, in its entirety or in part. The first scout image 402 and the second scout image 502 may be from a same imaging examination where the Z-axis reference origin is reset between acquisitions and/or from two different imaging examinations.

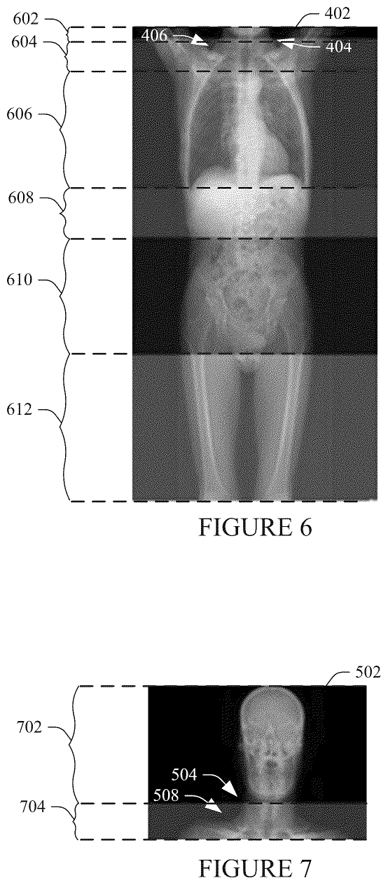

Returning to FIG. 3, the cumulative X-ray radiation dose profile generating module 158 includes a region detector 302. The region detector 302 is configured to detect a predetermined set of different anatomical regions of a patient in the scout images. For example, in one instance the predetermined set of different anatomical regions includes the head, the neck and shoulders, the abdomen, the pelvis, the legs, and the legs. In another instance, the predetermined set of different anatomical regions includes similar, different, more, less, etc. anatomical regions. FIG. 6 graphically depicts an example in which the region detector 302 detects a head region 602, a neck and shoulders region 604, a chest region 606, an abdomen region 608, a pelvic region 610 and a legs region 612 in the first scout image 402, and FIG. 7 graphically depicts an example in which the region detector 302 detects a head region 702 and a neck and shoulder region 704 in the second scout image 502.

In general, the patient will have their arms raised above their head for image acquisitions below the head, including the chest, abdomen and/or other anatomical regions, so that the arms are not in a path of the X-ray radiation beam, and at the sides of their body for image acquisitions of the head so that the arms are not in a path of the X-ray radiation beam. In FIGS. 4 and 6, the patient's arms are above their head for the body scout image 402, and in FIGS. 5 and 7, the patient's arms are at their sides for the head scout image 502. In other instances, e.g., where the patient is unable to position one or both of their arms, one or both arms might be included in the first scout image 402 and/or the second scout image 502. For sake of brevity and explanatory purposes, the arms of the patient generally are not considered in this example for the most part.

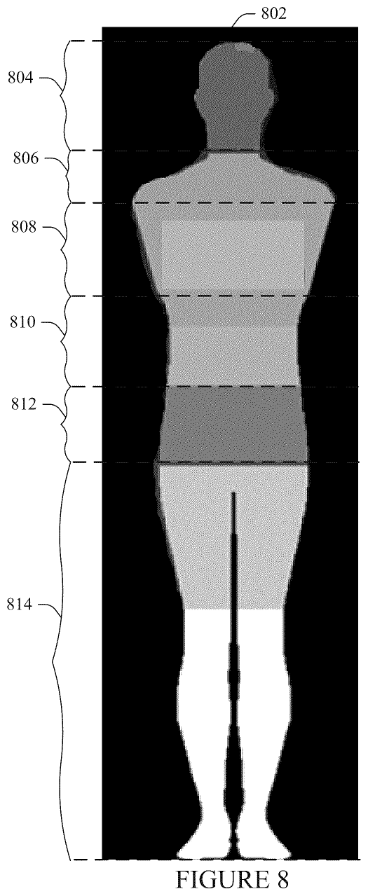

Returning to FIG. 3, the cumulative X-ray radiation dose profile generating module 156 further includes an overlapping region aligner 304. The overlapping region aligner 304 receives, as input, a human reference image that is pre-marked with a set of predetermined anatomical regions. An example human reference image 802 is graphically illustrated in FIG. 8. In this example, the human reference image 802 is pre-marked based on the predetermined set of different anatomical regions the anatomical regions discussed in connection with FIGS. 6 and 7. For example, the human reference image 802 includes a head region 804, a neck and shoulder region 806, a chest region 808, an abdomen region 810, a pelvic region 812, and a legs region 814. In another example, the human reference image 802 may include similar, different, more, less, etc. anatomical regions. In addition, in one instance, one or more of the anatomical regions of the human reference image 802 further includes graphical indicia indicative of one or more organs, one or more types of tissue, etc.

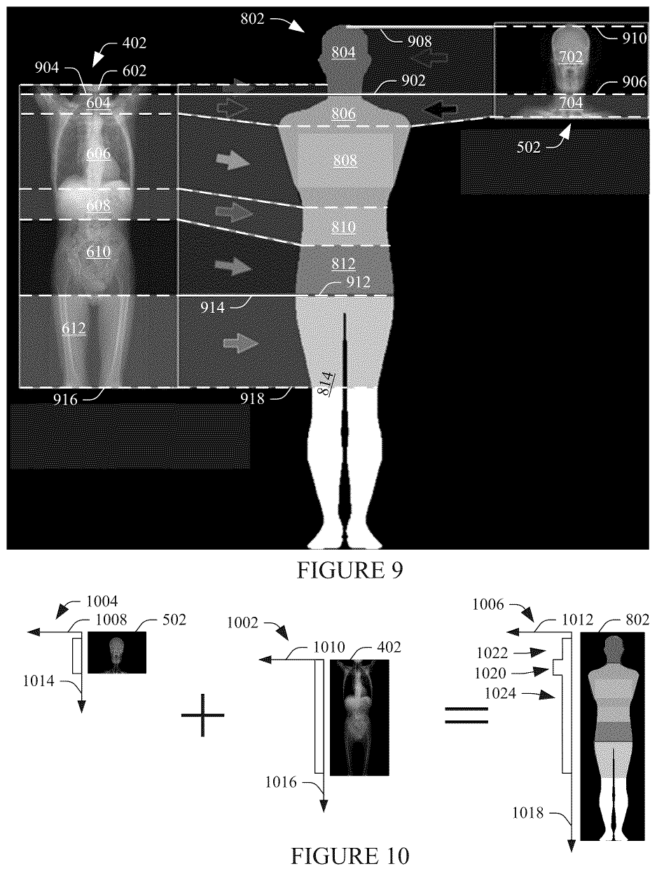

Returning to FIG. 3, the overlapping region aligner 304 is configured to align the first scout image 402 of the body and the detected anatomical regions 602, 604, 606, 608, 610 and 612 (FIG. 6) with the human reference image 802 of the body (FIG. 8) and to align the second scout image 502 of the head and the detected anatomical regions 702 and 704 (FIG. 7) with the human reference image 802 of the body (FIG. 8). More particularly, the overlapping region aligner 304 is configured to align the detected anatomical regions 602, 604, 606, 608, 610 and 612 in first scout image 402 respectively with the corresponding anatomical regions 804, 806, 808, 810, 812 and 814 of the human reference image 802 and the detected anatomical regions 702 and 704 in the second scout image 502 respectively with the corresponding anatomical regions 804 and 806 of the reference image 802. An example is graphically depicted in FIG. 9.

For this, in one instance, an anatomical boundary that is included in the first scout image 402, the second scout image 502 and the human reference image 802 is identified. Since both the first scout image 402 and the second scout image 502 include the head (the head 602 and the head 702) and the neck and shoulders (the neck and shoulders 604 and the neck and shoulders 704), a head/neck and shoulder boundary 902 is identified in the reference image 802, a head/neck and shoulder boundary 904 is identified on the first scout image 402 as a shared or common boundary between the head 602 and the neck and shoulders 604, and a head/neck boundary 906 is identified on the second scout image 502 as a shared or common boundary between the head 702 and the neck and shoulders 704. The first scout image 402, the second scout images 502 and the human reference image 802 are aligned such that their respective boundaries 902, 904 and 906 are aligned. In one instance, this ensures that overlapping regions are matched accurately across scout images 402 and 502 and the human reference image 802.

Returning to FIG. 3, the cumulative X-ray radiation dose profile generating module 156 further includes an anatomical region expander 306. The anatomical region expander 306 is configured to expand anatomical regions between the human reference image 802 and the scout images 402 and 502, e.g., by deduction or the like for full region in the scout images 402 and 502 and other approaches (e.g., based on average dimensions, etc.) for truncated regions in the scout images 402 and 502.

For example, in one instance, for the second scout image 502, a 908 top of the head 804 in the human reference image 802 (FIG. 9) is identified and a top 910 of the head 702 in the second scout image 502 is identified, e.g., by scanning the second scout image 502 top to bottom, bottom to top, and/or otherwise. The top 910 of the head 702 in the second scout image 502 is then aligned with the top 908 of the human reference image 802 so that the dose map of the scout image 502 can be linked with the human reference image 802. In instances in which the second scout image 502 does not include the entire head, the missing portion can be estimated.

For example, where the size of the patient is known, the DICOM file includes a size of each scout pixel (e.g., 1 pixel=1 mm), and such information can be determined from the DICOM file. If the size of the patient is unknown, the size can be estimated based on the size of each available anatomical region and average human ratio. n instances in which the full region and the size of the patient are both unknown, the patient size can be estimated as a mean size for an adult or a child, a male or a female, an age of the patient, and/or other available patient information that can be utilized to estimate a size of a patient. Other approaches are contemplated herein. When a region is not fully available on the scout image (i.e., a region is cropped, truncated, etc. such as the head and legs regions in FIG. 6), aligning it may be more complex than when a region is fully available on the scout (i.e., the chest, the abdomen, the pelvis, etc. in FIG. 6).

For the first scout image 402, features are similarly identified, e.g., by scanning the second scout image 502 from top to bottom, bottom to top, and/or otherwise. In one instance, a starting position of a last visible anatomical region is identified and used to align the first scout image 402 and the human reference image 802. In the illustrated example, the last visible anatomical region in the first scout image 402 is in the legs region 612. As such, a top 912 of the legs region 612 (e.g., a boundary between the legs region 612 and the pelvic region 610) is identified in the first scout image 402, and a top 914 of the legs region 814 (e.g., a boundary between the legs region 814 and the pelvic region 812) is identified in the human reference image 802 (FIG. 9). The top 912 of the legs region 612 in the first scout image 402 is then aligned with the top 914 of the legs region 814 in the human reference image 802.

In one instance, this ensures that the first scout image 402 of the body is aligned with the dimensions of the human reference image 802 regardless of where the first scout image 402 of the body is truncated. For a cropped, truncated, etc. area, e.g., the region below the legs region 612 of the first scout image 402, an end position is determined based the human reference image 802, which is considered a reference. For example, in one instance the projection of a bottom 916 of the legs regions 612 (which is truncated in that, e.g., the knees, the ankles, the feet, etc. are not in the legs region 612) to the human reference image 802 is based on a ratio of the legs regions 612 to the legs region 814 of the human reference image 802. In this example, a projection 918 on the human reference image 802 is aligned horizontally with the bottom 916 of the legs regions 612. In another instance the projection 918 may be higher or lower than the bottom 916 of the legs regions 612, depending on the ratio.

The remaining corresponding regions are then mapped. For instance, a bottom of the neck and shoulders region 704 in the second scout image 502 does not align with a bottom of the neck and shoulders region 806 in the human reference image 802, but the bottom of the neck and shoulders region 704 in the second scout image 502 maps to the bottom of the neck and shoulders region 806 in the human reference image 802. Likewise, a bottom of the neck and shoulders region 604/a top of the chest region 606 in the first scout image 402 does not align with the bottom of the neck and shoulders region 806/top of the chest region 808 in the human reference image 802, but the bottom of the neck and shoulders region 604/top of the chest region 606 in the first scout image 402 maps to the bottom of the neck and shoulders region 806/ top of the chest region 808 in the human reference image 802.

A bottom of the chest region 606/top of the abdomen region 608 the first scout image 402 does not align with a bottom of the chest region 808/top of the abdomen region 810 in the human reference image 802, but the bottom of the chest region 606/top of the abdomen region 608 the first scout image 402 maps to the bottom of the chest region 606 in the human reference image 802. A top of the abdomen region 608 of the first scout image 402 does not align with a top of the abdomen region 810 in the human reference image 802, but aligns with the bottom of the chest region 808/top of the abdomen region 810 in the human reference image 802. A bottom of the abdomen region 608/top of the pelvic region 610 of the first scout image 402 does not align with a bottom of the abdomen region 810/top of the pelvic region 812 in the human reference image 802, but the bottom of the abdomen region 608/top of the pelvic region 610 of the first scout image 402 maps to the bottom of the abdomen region 810/top of the pelvic region 812 in the human reference image 802.

A bottom of the pelvic region 610 the first scout image 402 aligns with a bottom of the pelvic region 812 in the human reference image 802 since the human reference image 802 was scaled to align the top of the legs 814 with the top 914 of the legs 612 of the fist scout image 402, and the bottom of the pelvic region 812 is the top of the legs 814 of the human reference image 802 and the bottom of the pelvic region 610 is the top of the legs 612 of the first scout image 402. A bottom of the legs 612 (which is cropped in the example) of the first scout image 402 aligns a bottom of the legs 814 of the human reference image 802.

Returning to FIG. 3, the cumulative X-ray radiation dose profile generating module 158 further includes a dose projector 308. The dose projector 308 is configured to project the X-ray radiation dose from all the identified anatomical regions of all the scout images (i.e., the first scout 402 and the second scout 502 in this example) aligned with the human reference image 802 to the corresponding regions of the human reference image 802. For example, the X-ray radiation dose for the head region 702 of the second scout image 502 and the X-ray radiation dose for the head region 602 of the first scout image 402 are both projected on the head region 804 of the human reference image 802, as shown in FIG. 9. Likewise, the X-ray radiation dose for neck and shoulder region 704 of the second scout image 502 and the X-ray radiation dose for neck and shoulder region 604 of the second scout image 502 are both projected on the neck and shoulder region 806 of the human reference image 802, as shown in FIG. 9.

Returning to FIG. 3, the cumulative X-ray radiation dose profile generating module 156 further includes a summer 310. The summer 310 is configured to sum the X-ray radiation dose projected from each anatomical region of each scout image to each anatomical region of the human reference image 802 and provide a cumulative X-ray radiation dose over time for each anatomical region. In general, the cumulative X-ray radiation dose can be determined based on CTDI, SSDE, and the like. For the cumulative X-ray radiation dose, the summation of CTDI's considers the type of the phantom. For example, a CTDI computed based on a head phantom cannot be summed with a CTDI computed based on a body phantom. However, SSDE's can be summed without considering the types of the phantom. That is, an SSDE computed based on a head phantom can be summed with an SSDE computed based on a body phantom.

A non-limiting example of the summing is graphically illustrated in FIG. 10, which includes the first scout image 402 along with a corresponding dose plot 1002, the second scout image 502 along with a corresponding dose plot 1004, and the human reference image 802 along with a cumulative dose plot 1006, which includes a summation of the plots 1002 and 1004. The plots 1002, 1004 and 1006 respectively include first axes 1008, 1010 and 1012 that represent dose (e.g., in units of milligray (mGy)) and second axes 1014, 1016 and 1018 that represent a longitudinal axis of the patient. The dose values in the plots 1002 and 1004 are the same in this example for explanatory purposes. However, the dose values in the plots 1002 and 1004 can be different. The cumulative dose plot 1006 includes a region 1020 that represents an overlapping irradiated anatomy of the first and second scout images 402 and 502 and regions 1022 and 1024 where there was no overlap. It is to be appreciated that the dose of each of the dose plots 1002 and 1004 may correspond to one or more volume scans.

In one instance, the human reference image 802 and the cumulative dose plot 1006 are visually presented to the user. In some instances, the first scout image 402, the dose plot 1002, the second scout image 502, and/or the dose plot 1004 are additionally and/or alternatively visually presented. In instances with more than two scout images, the cumulative dose plot 1006 will include a summation of dose values of more than two scout images. The cumulative X-ray radiation dose profile with the reference image 802 and the cumulative dose plot 1006 provide an X-ray radiation dose map, mapping the X-ray radiation dose of each scout image to anatomical regions of the human reference image 802.

As discussed herein, not all of the scout images for a patient will include a same Z-axis reference origin, whether part of a same imaging examination of the patient and/or part of different imaging examinations, for the patient performed over time. In this example, the first scout image 402 and the second scout image 502 do not have the same Z-axis reference origin. With a configuration that does not employ and/or include the cumulative X-ray radiation dose profile generating module 156, there currently is no effective approach for aggregating the X-ray radiation dose for the anatomical regions of the first scout image 402 and the second scout image 502. As a consequence, a patient may be exposed to unnecessary and/or untracked X-ray radiation dose corresponding to overlapping irradiated regions. The cumulative X-ray radiation dose profile generating module 156 provides an effective approach for aggregating the X-ray radiation dose for the anatomical regions of the first scout image 402 and the second scout image 502 to produce a cumulative X-ray radiation dose profile over time. As such, patient X-ray radiation dose and/or overlapping irradiated regions can be identified and/or tracked, and mitigated for future scans, where appropriate.

Turning now to FIG. 11, another example of the cumulative X-ray radiation dose profile generating module 156 is schematically illustrated. This example is discussed in connection with FIGS. 11, 12, 13, 14, 15, 16, 17, 18, 19, 20, 21, 22 and 23.

Initially referring to FIG. 11, the cumulative X-ray radiation dose profile generating module 156 receives, as input, multiple scout images (some having different Z-axis reference origins) along with individual X-ray radiation dose profiles for each of the scout images. Examples are graphically depicted and discussed in connection with FIGS. 4 and 5, which respectively depict the first scout image 402 of the body and the second scout image 502 of the head. Again, the first scout image 402 and the second scout image 502 may be from a same imaging examination where the Z-axis reference origin is reset between acquisitions and/or from two different imaging examinations. Other examples may include more scout images, scout images of different regions of the patient, multiple scout images of a same region of the patient, etc.

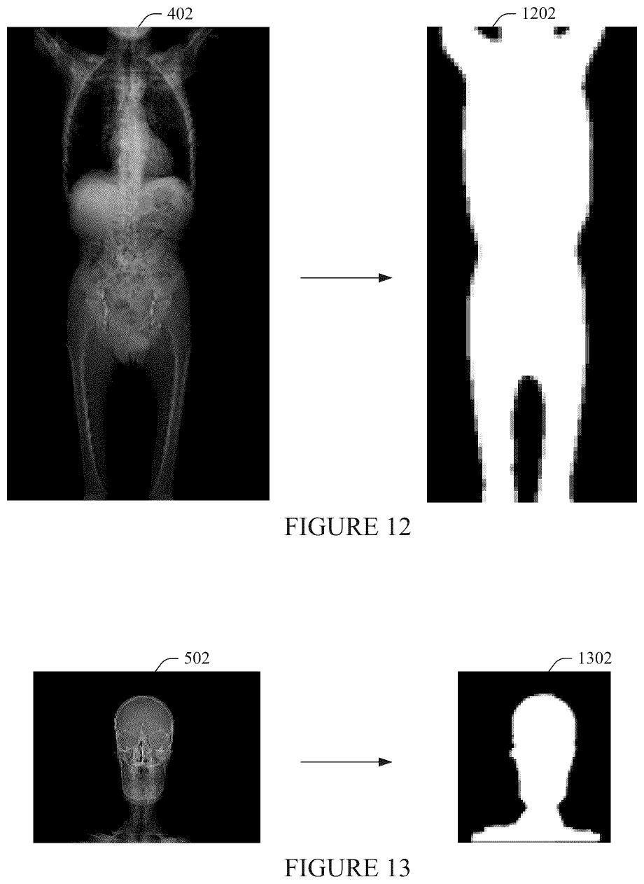

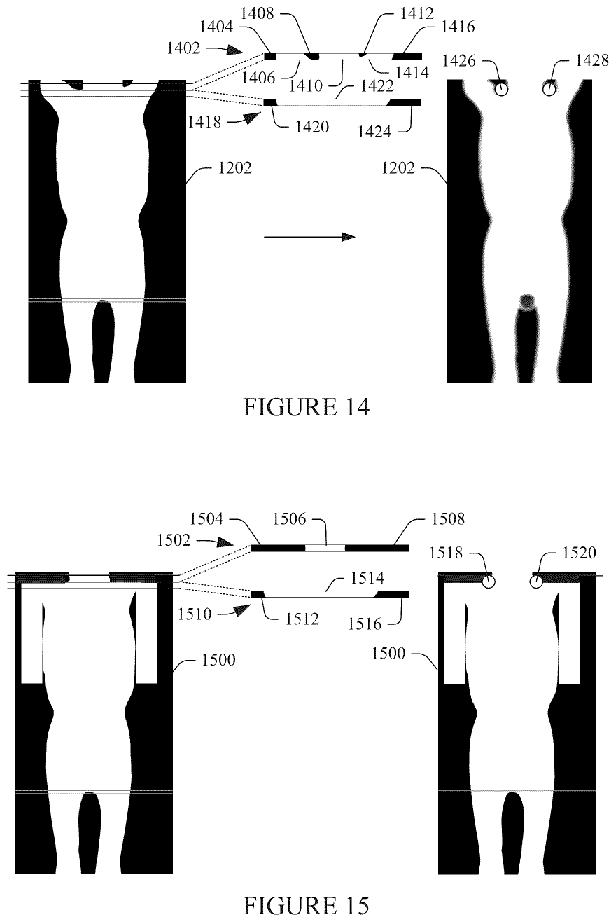

The cumulative X-ray radiation dose profile generating module 156 includes a mask generator 1102. The mask generator 1102 is configured to generate a mask image for each input scout image, e.g., the first mask image 1202 (FIG. 12) for the first scout image 402 (FIG. 4) and the second mask image 1302 (FIG. 13) for the second scout image 502 ((FIG. 5), in this example. Briefly turning to FIGS. 12 and 13, FIG. 12 graphically depicts the first scout image 402 and a corresponding first mask image 1202 therefore, and FIG. 13 graphically depicts the second scout image 502 and a corresponding second mask image 1302 therefor. Known and/or other approaches can be utilized to generate the first mask image 1202 and the second scout image 502. For example, a voxel and/or pixel thresholding approach can be utilized to generate binary masks of outer perimeters or contours of the first scout image 402 and the second scout image 502.

Returning to FIG. 11, the cumulative X-ray radiation dose profile generating module 156 further includes a marker positioner 1104. The marker positioner 1104 is configured to place anatomical markers corresponding to a predetermined set of anatomical locations on the mask images, including the first mask image 1202 and the second mask image 1302. In this example, the predetermined set of anatomical locations include the top of the head, the top of the shoulders and the crotch. Known and/or other approaches can be utilized to identify the set of anatomical locations in the first mask image 1202 and the second mask image 1302. Example approaches are described next in connection with FIGS. 14, 15, 16, 17 and 18.

Beginning with FIG. 14, first mask image 1202 (which corresponds to the first scout image 402, which includes the head region 602, the neck and shoulder region 604, the chest region 606, the abdomen region 608, the pelvic region 610 and the legs region 612) is evaluated to identify the top of the shoulders. For the top of the shoulders, the approach will vary depending on whether the arms of the patient are raised above the head of the patient or are at sides of the patient. The position of the arms of the patient can be obtained from metadata for the first scout image 402, metadata for the first mask image 1202, the imaging protocol selected for the volume scan, an auto-identification algorithm, via entry by a user, and/or otherwise. With one approach, a pair of consecutive (neighboring, adjacent, etc.) rows at a transition between just above the shoulders and at the shoulders is identified.

For arms up, the row directly above the shoulders will include the following pattern: a shorter black region (outside of the patient), a shorter white region (one arm), a shorter black region (between the one arm and the neck), a shorter white region (the neck), a shorter black region (between the other one arm and the neck), a shorter white region (the other arm), and a shorter black region (outside of the patient). For arms down, the row directly above the shoulders will include the following pattern: a longer black region (outside of the patient), a shorter white region (the neck), and a longer black region (outside of the patient). For both arms above the head and arms at the sides, the row at the shoulders will include the following pattern: a shorter black region (outside of the patient), a longer white region (the shoulders), and a shorter black region (outside of the patient).

With the scout image 402 and hence, the first mask image 1202, the arms of the patient are raised above their head. The first mask image 1202 is evaluated to determine pixel patterns across rows of pixels. A first row 1402 corresponds to a first row above the shoulders. The first row 1402 includes a first region 1404 outside of the patient (i.e., black pixels), a second region 1406 corresponding to an arm of the patient (i.e., white pixels), a third region 1408 corresponding to a region between one arm and the neck of the patient (i.e., black pixels), a fourth region 1410 corresponding to a neck of the patient (i.e., white pixels), a fifth region 1412 corresponding to a region between the other arm and the neck of the patient (i.e., black pixels), a sixth region 1414 corresponding to the other arm of the patient (i.e., white pixels), and a seventh region 1416 outside of the patient (i.e., black pixels).

An adjacent row 1418 corresponds to the shoulders of the patient and includes a first region 1420 outside of the patient (i.e., black pixels), a second region 1422 corresponding shoulders of the patient (i.e., white pixels), and a third region 1424 outside of the patient (i.e., black pixels). The marker positioner 1104 positions a first position marker 1426 on the first mask image 1202 at a location corresponding to the region between one arm and the neck of the patient (the third region 1408) and a second position marker 1428 on the first mask image 1202 at a location corresponding to the region between the other arm and the neck of the patient (the third region 1412).

Briefly turning to FIG. 15, an example approach for when the arms are at the sides of patient is illustrated. A mask image 1500 is evaluated to determine pixel patterns across rows of pixels. A first row 1502 corresponds to a first row above the shoulders. The first row 1502 includes a first longer region 1504 outside of the patient (i.e., black pixels), a second shorter region 1506 corresponding to a neck of the patient (i.e., white pixels), and a third longer region 1508 outside of the patient (i.e., black pixels). An adjacent row 1510 corresponds to the shoulders of the patient and includes a first shorter region 1512 outside of the patient (i.e., black pixels), a second longer region 1422 corresponding to shoulders the patient (i.e., white pixels), and a third shorter region 1424 outside of the patient (i.e., black pixels). The marker positioner 1104 positions a first position marker 1518 on the first mask image 1202 at a location corresponding to the shoulders and a second position marker 1520 on the first mask image 1202 at a location corresponding to the shoulders.

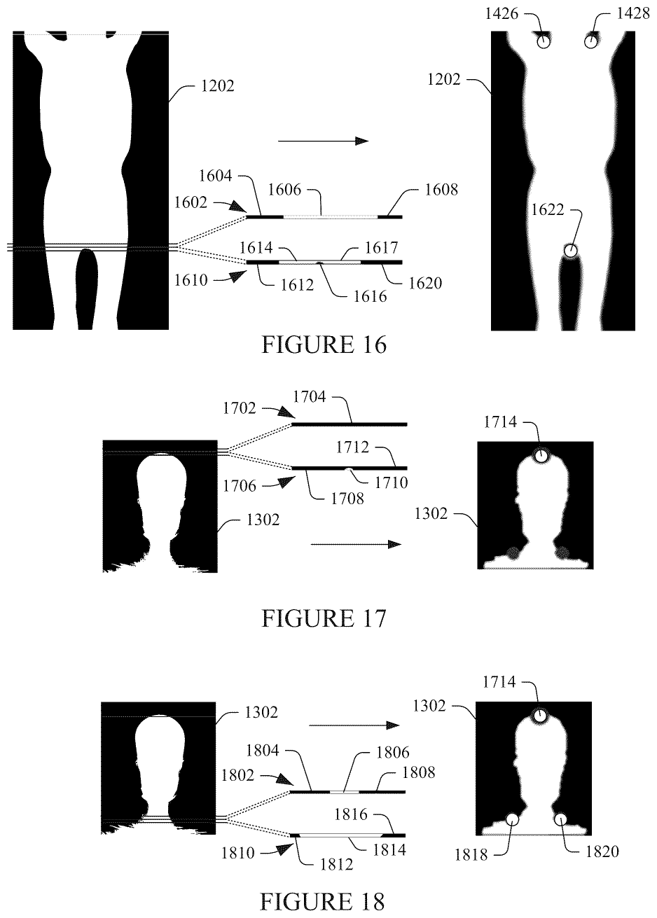

Moving to FIG. 16, the first mask image 1202 (which corresponds to the first scout image 402, which includes the head region 602, the neck and shoulder region 604, the chest region 606, the abdomen region 608, the pelvic region 610 and the legs region 612) is evaluated to identify the crotch. The marker positioner 1104 evaluates the first mask image 1202 and identifies a pair of consecutive rows at a transition between above the crotch and at the crotch. Similar to the shoulders, in one instance, the marker positioner 1104 identifies pixel patterns across rows of pixels.

A first row 1602 corresponds to a first row above the crotch. The first row 1602 includes a first region 1604 outside of the patient (i.e., black pixels), a second region 1606 corresponding to the pelvis of the patient (i.e., white pixels), and a third region 1508 outside of the patient (i.e., black pixels). An adjacent row 1610 corresponds to the crotch of the patient and includes a first region 1612 outside of the patient (i.e., black pixels), a second region 1614 corresponding to a leg of the patient (i.e., white pixels), a third region 1616, a fourth region 1617 corresponding to the other leg of the patient (i.e., white pixels), and a fifth region 1620 outside of the patient (i.e., black pixels). The marker positioner 1104 positions a crotch position marker 1622 on the first mask image 1202 at a location based on the pair of rows 1602 and 1604.

Next at FIGS. 17 and 18, the second mask image 1302 (which corresponds to the second scout image 502, which includes the head region 702 and the neck and shoulder region 704) is evaluated to identify a top of the head and a top of the shoulders. For the top of the head, the marker positioner 1104 evaluates the second mask image 1302 and identifies a pair of consecutive rows that are at a transition between above the head (and outside of the patient) and at the head. For the shoulders, the marker positioner 1104 evaluates the second mask image 1302 and identifies a pair of consecutive rows that are at a transition between above the shoulders and at the shoulders. Similar to the first mask image 1202, the marker positioner 1104 identifies pixel patterns across rows of pixels.

Beginning with FIG. 17, for the top of the head, a first row 1702 corresponds to a first row above the head. The first row 1702 includes a first region 1704 outside of the patient (i.e., all black pixels). An adjacent row 1706 corresponds to the head of the patient and includes a first region 1708 outside of the patient (i.e., black pixels), a second region 1710 corresponding to the head of the patient (i.e., some white pixels), and a third region 1712 outside of the patient (i.e., black pixels). The marker positioner 1104 positions a top of head position marker 1714 on the first mask image 1202 at a location based on the pair of rows 1702 and 1706.

At FIG. 18, for the shoulders, a first row 1802 corresponds to a first row above the head. The first row 1802 includes a first region 1804 outside of the patient (i.e., black pixels). A first row 1802 corresponds to a first row above the shoulders. The first row 1802 includes a first longer region 1804 outside of the patient (i.e., black pixels), a second shorter region 1806 corresponding to a neck of the patient (i.e., white pixels), and a third longer region 1808 outside of the patient (i.e., black pixels). An adjacent row 1810 corresponds to the shoulders of the patient and includes a first shorter black region 1812 outside of the patient (i.e., black pixels), a second longer region 1814 corresponding to shoulders the patient (i.e., white pixels), and a third shorter region 1816 outside of the patient (i.e., black pixels). The marker positioner 1104 positions a first shoulder position marker 1818 and a second position marker 1820 on the first mask image 1202 at locations based on the pair of rows 1802 and 1810.

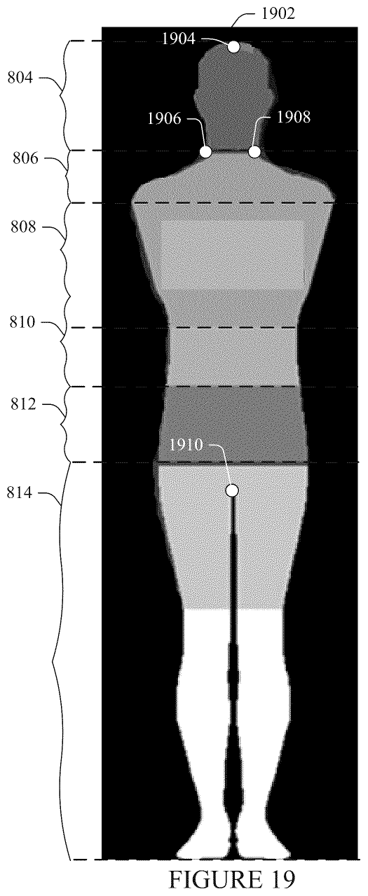

Returning to FIG. 11, the cumulative X-ray radiation dose profile generating module 156 further includes a reference to mask aligner 1106. The reference to mask aligner 1106 receives, as input, a human reference image that is pre-marked with location makers for the top of the head, the shoulder and the crotch and segmented into a plurality of anatomical regions. An example reference image 1902 is graphically illustrated in FIG. 19. In this example, the human reference image 1902 includes the reference image 802 (including the head region 804, the neck/shoulder region 806, the chest region 808, the abdomen region 810, the pelvic region 812, and the legs region 814), along with a top of head marker 1904, a first side shoulder marker 1906, a second side shoulder marker 1908, and a crotch marker 1910.

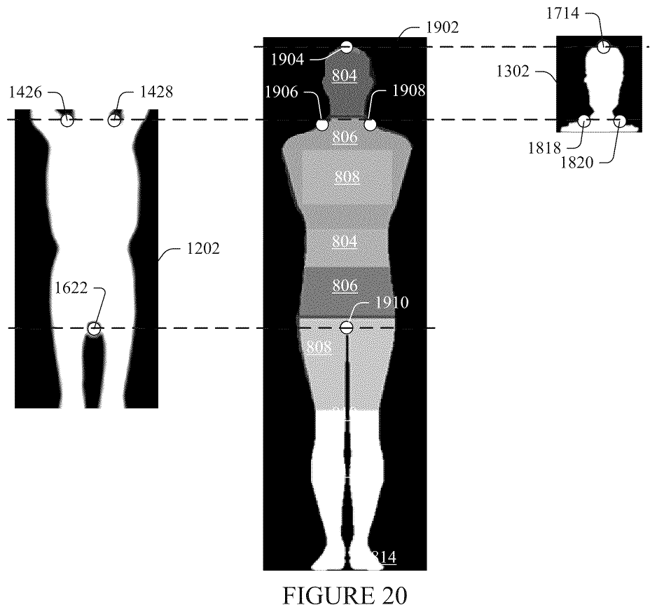

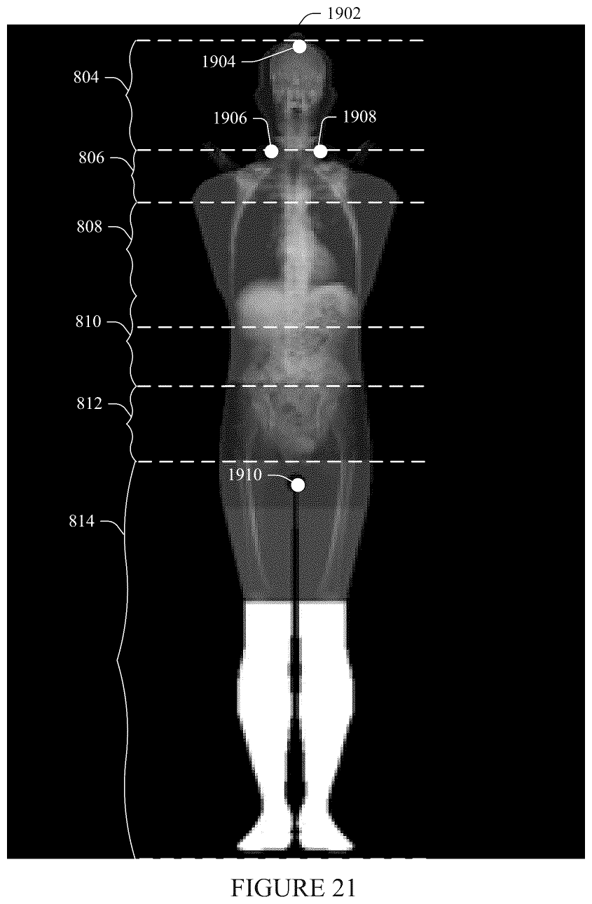

Returning to FIG. 11, the reference to mask aligner 1106 is configured to align the first mask image 1202 and the second mask image 1302 with the human reference image 1902 based on the markers. An example is illustrated in FIG. 20. The shoulder markers 1426 and 1428 and the crotch marker 1622 of the first mask image 1202 are aligned with the shoulder markers 1906 and 1908 and the crotch marker 1910 of the human reference image 1902. A size of the first mask image 1202 is adjusted as need to spatially match the markers 1426, 1428 and 1622 of the first mask image 1202 respectively with the markers 1906, 1908 and 1910 of the human reference image 1902. The top of the head marker 1714 and the shoulder markers 1818 and 1820 of the second mask image 1302 are aligned with the top of the head marker 1904 and the shoulder markers 1906 and 1908 of the human reference image 1902. A size of the second mask image 1302 is adjusted as needed to spatially match the markers 1714, 1818 and 1820 of the second mask image 1302 respectively with the markers 1904, 1906 and 1908 of the human reference image 1902.

Returning to FIG. 11, the cumulative X-ray radiation dose profile generating module 156 further includes a reference to mask mapper 1108. The reference to mask mapper 1108 is configured to map corresponding anatomical regions of the human reference image 1902 to the first mask image 1202 aligned with the human reference image 1902 and the second mask image 1302 aligned with the human reference image 1902. The head region 804, the neck and shoulder region 806, the chest region 808, the abdomen region 810, the pelvic region 812 and the legs region 814 of the human reference image 1902 are mapped onto the first mask image 1202, and the head region 804 and the neck and shoulder region 806 of the human reference image 1902 are mapped onto the second mask image 1302.

Returning to FIG. 11, the cumulative X-ray radiation dose profile generating module 156 further includes a scout to reference image matcher 1110. The scout to reference image matcher 1110 is configured to apply the anatomical regions to be able to correctly sum the doses. Since the first mask image 1202 and the first scout image 402 are a same size, and the second mask image 1302 and the second scout image 502 are a same size, the anatomical regions (i.e., the head region 804, the neck and shoulder region 806, the chest region 808, the abdomen region 810, the pelvic region 812, and the legs region 814 from the first mask image 1202 map to the first scout image 402 and from the second mask image 1302 map to the second scout image 502.

The cumulative X-ray radiation dose profile generating module 156 further includes a scout and reference combiner 1112. The scout and reference combiner 1112 is configured to combine the anatomical regions of the first scout image 402 from FIG. 4 and the anatomical regions of the second scout image 502 from FIG. 5 with corresponding anatomical regions of the human reference image 1902 of FIG. 19. An example is graphically illustrated in FIG. 21, which graphically depicts the human reference image 1902 with the first scout image 402 and the second scout image 502 superimposed or overlaid thereover and matched to the anatomical regions (i.e., the head region 804, the neck and shoulder region 806, the chest region 808, the abdomen region 810, the pelvic region 812, and the legs region 814).

The cumulative X-ray radiation dose profile generating module 156 further includes a summer 1114. The summer 1114 is configured to sum, for each of the head region 804, the neck and shoulder region 806, the chest region 808, the abdomen region 810, the pelvic region 812, and/or the legs region 814, X-ray radiation dose contributions from the first scout image 402 and the second scout image 502. The approach described in connection with FIGS. 3 and 10 can be utilized, where the cumulative dose plot 1006 includes a summation of the plots 1002 and 1004, and the region 1020 represents an overlapping irradiated anatomy of the first and second scout images 402 and 502.

Similar to the example described in connection with FIGS. 3 and 10, in one instance, the human reference image 1902 and the cumulative dose plot 1006 are visually presented to the user. In some instances, the first scout image 402 the dose plot 1002 and the second scout image 502 and the dose plot 1004 are additionally and/or alternatively visually presented. In instances with more than two scout images, the cumulative dose plot 1006 will include a summation of dose values of more than two scout images. The cumulative X-ray radiation dose profile with the reference image 802 and the cumulative dose plot 1006 provide an X-ray radiation dose map, mapping the X-ray radiation dose of each scout image to anatomical regions of the human reference image 1902.

Again, not all of the scout images for a patient will include a same Z-axis reference origin, whether part of a same imaging examination of the patient and/or part of different imaging examinations for the patient performed over time. In this example, the first scout image 402 and the second scout image 502 do not have the same Z-axis reference origin. With a configuration that does not employ and/or include the cumulative X-ray radiation dose profile generating module 156, there currently is no effective approach for aggregating the X-ray radiation dose for the anatomical regions of the first scout image 402 and the second scout image 502. As a consequence, a patient may be exposed to unnecessary and/or untracked X-ray radiation dose corresponding to overlapping irradiated regions. The cumulative X-ray radiation dose profile generating module 156 provides an effective approach for aggregating the X-ray radiation dose for the anatomical regions of the first scout image 402 and the second scout image 502, providing a cumulative X-ray radiation dose profile over time. As such, patient X-ray radiation dose and/or overlapping irradiated regions can be identified and/or tracked, and mitigated for future scans, where appropriate.

It is to be appreciated that the approach described in connection with FIG. 3-10 and the approach described in connection with FIGS. 11-23 are complementary and can be utilized individually and/or in combination. For example, in one instance only the approach described in connection with FIG. 3-10 is utilized, in another instance, only the approach described in connection with FIG. 11-23 is utilized, and yet in another instance both the approach described in connection with FIG. 3-10 and in connection with FIG. 11-23 are utilized. Where both approaches are utilized, the results can be compared to determine whether they provide a similar result, which verifies the results, or contradictory results which may indicate that at least one of the two approaches was not successful. In addition, the approach described in connection with FIG. 11-23 can be utilized to add more landmarks to the segmentation of the approach described in connection with FIG. 3-10.

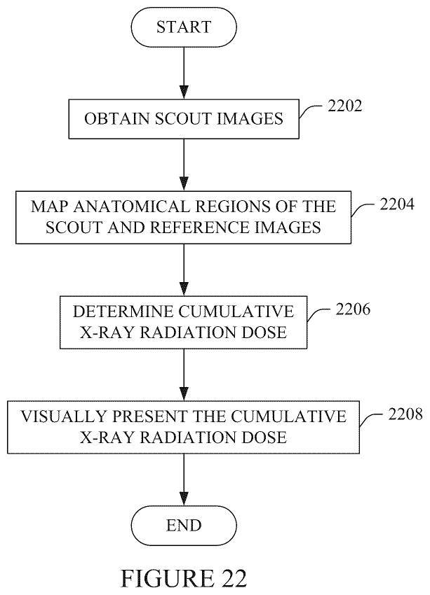

FIG. 22 illustrates a non-limiting example of a flow chart for a method of determining a cumulative patient X-ray radiation dose profile that is based on scout images independent of Z-axis reference origins. It is to be appreciated that the ordering of the acts in the method is not limiting. As such, other orderings are contemplated herein. In addition, one or more acts may be omitted, and/or one or more additional acts may be included.

At 2202, at least a first scout image for a patient and an X-ray radiation dose profile therefore and a second scout image for the patient and an X-ray radiation dose profile therefore are obtained, as described herein and/or otherwise. Examples of such images include the first scout image 402 (FIG. 4) and the second scout image 502 (FIG. 5). In another instance, other scout images for the patient are also obtained, including scout images corresponding to a same and/or different imaging examination, a same and/or different Z-axis reference origin, and/or with overlapping, contiguous and/or non-overlapping, and non-contiguous anatomical regions.

At 2204, anatomical regions of the scout and human reference images are mapped, as described herein and/or otherwise. For example, in one instance the scout images automatically assembled on a pre-marked reference image. In another instance, anatomical regions visible on the scout images are classified by associating them with corresponding pre-marked regions of a reference image using common region boundaries. In general, this step repositions scout images based on region boundaries on the images, without depending on the geometric coordinates provided by the imaging system, which may be erroneous or incomplete.

At 2206, the X-ray radiation dose of the scout images are summed based on the mapping, generating a cumulative X-ray radiation dose profile over time that includes a cumulative X-ray radiation dose map, as described herein and/or otherwise. An example is graphically illustrated FIG. 10, e.g., the human reference image 802 (or the human reference image 1902) with the cumulative X-ray radiation dose map 1006 for the first scout images 402 and the second scout image 502.

At 2208, the cumulative X-ray radiation dose map (with or without other information) is displayed to a user and/or otherwise utilized. An example visual presentation is graphically illustrated FIG. 10, e.g., the human reference image 802 (or the human reference image 1902) with the cumulative X-ray radiation dose map 1006 for the first scout image 402 and the second scout image 502.

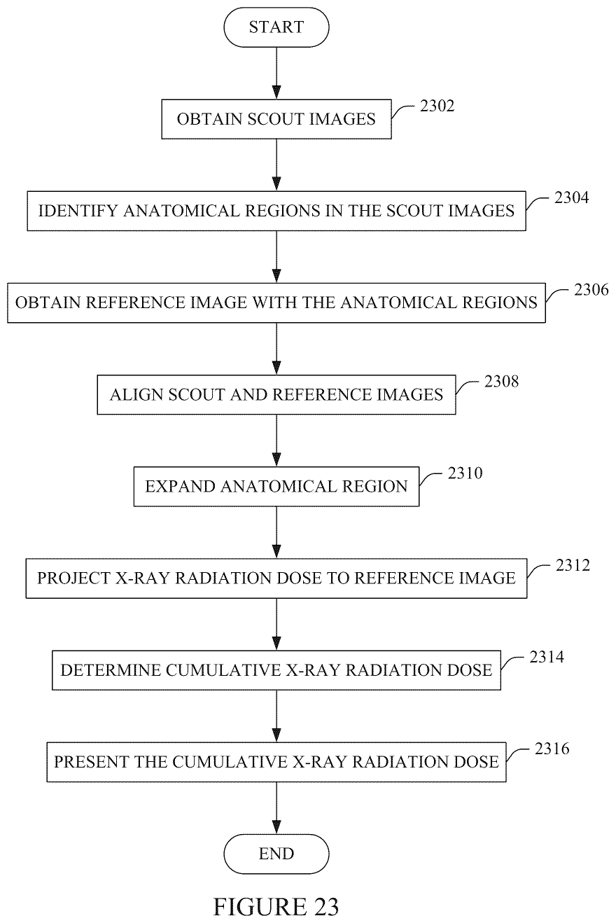

FIG. 23 illustrates a non-limiting example of a flow chart for a method of determining a cumulative patient X-ray radiation dose profile that is based on scout images independent of Z-axis reference origins. It is to be appreciated that the ordering of the acts in the method is not limiting. As such, other orderings are contemplated herein. In addition, one or more acts may be omitted, and/or one or more additional acts may be included.

At 2302, at least a first scout image for a patient and an X-ray radiation dose profile therefore and a second scout image for the patient and an X-ray radiation dose profile therefore are obtained, as described herein and/or otherwise. Examples of such images include the first scout image 402 (FIG. 4) and the second scout image 502 (FIG. 5). In another instance, other scout images for the patient are also obtained, including scout images corresponding to a same and/or different imaging examination, a same and/or different Z-axis reference origin, and/or with overlapping, contiguous and/or non-overlapping, and non-contiguous anatomical regions.

At 2304, predetermined set of anatomical regions are identified in each of the first scout image and the second scout image, as described herein and/or otherwise. Examples include the first scout image 402 with the head region 602, the neck and shoulder region 604, the chest region 606, the abdomen region 608, the pelvic region 610, and the legs region 612 (FIG. 6) and the second scout image 502 with the head region 702 and the neck and shoulder region 704 (FIG. 7). In another instance, the same, more or less anatomical regions are delineated.

At 2306, a reference image of a human, pre-marked with based on the predetermined set of anatomical regions, is obtained, as described herein and/or otherwise. An example includes the human reference image 802, which includes the head region 804, the neck and shoulder region 806, the chest region 808, the abdomen region 810, the pelvic region 812, and the legs region 814.

At 2308, overlapping regions of the first scout image and the second scout image are aligned with corresponding region of the human reference image, as described herein and/or otherwise. An example is graphically illustrated in FIG. 9, which depicts the first scout image 402 aligned with the human reference image 802 based on a head/neck and shoulders transition region and a pelvis/legs transition region, and the second scout image 502 aligned with the human reference image 802 based on a top of the head/outside the body transition and a head/neck and shoulders transition region.

At 2310, anatomical regions expanded between the scout images and the human reference, as described herein and/or otherwise. For example, anatomical regions can be expanded by deduction or the like for full region in the scout images 402 and 502 and other approaches (e.g., based on average dimensions, etc.) for truncated regions in the scout images 402 and 502. An example is graphically illustrated in FIG. 9.

At 2312, the X-ray radiation dose of the first scout image and the X-ray radiation dose of the second scout image are projected onto the human reference image based on the anatomical regions, as described herein and/or otherwise. An example is graphically illustrated in FIG. 9, which depicts the X-ray radiation dose of each of the anatomical regions of the first scout image 402 (i.e., 602, 604, 606, 608, 610 and 612) projected onto the corresponding anatomical regions of the human reference image 802, and each of the anatomical regions of the second scout image 502 (i.e., 702 and 704) projected onto the corresponding anatomical regions of the human reference image 802.

At 2314, the X-ray dose of the first scout image and the X-ray dose of the second scout image projected onto the human reference image are summed, generating a cumulative X-ray radiation dose profile over time that includes a cumulative X-ray radiation dose map, as described herein and/or otherwise. An example is graphically illustrated FIG. 10, e.g., the human reference 802 with the cumulative X-ray radiation dose map 1006 for the first scout image 402 and the second scout image 502.

At 2316, the cumulative X-ray radiation dose map (with or without other information) is displayed to a user and/or otherwise utilized. An example visual presentation is graphically illustrated FIG. 10, e.g., the human reference 802 with the cumulative X-ray radiation dose map 1006 for the first scout image 402 and the second scout image 502.

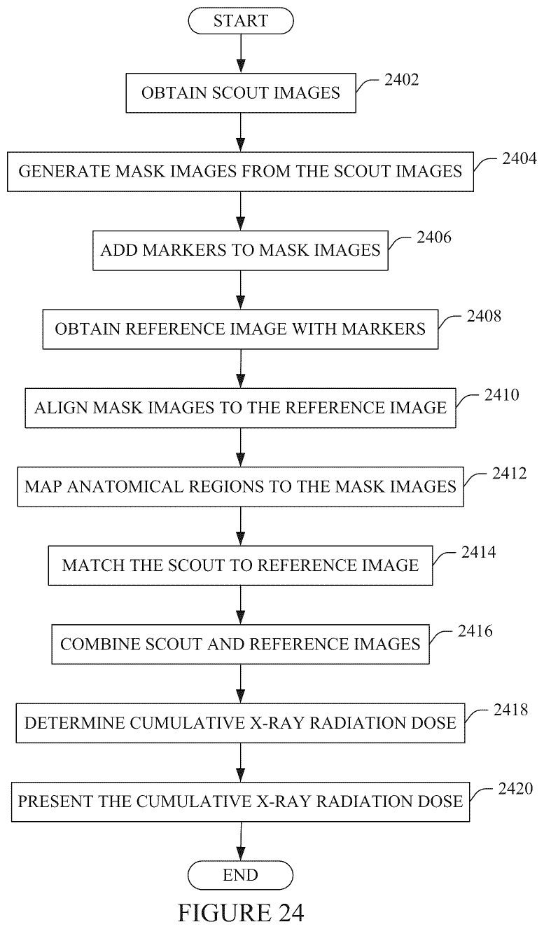

FIG. 24 illustrates a non-limiting example of a flow chart for a method of determining a cumulative patient X-ray radiation dose profile that is based on scout images with independent of reference origins. As such, other orderings are contemplated herein. In addition, one or more acts may be omitted, and/or one or more additional acts may be included.

At 2402, at least a first scout image for a patient and an X-ray radiation dose profile therefore and a second scout image for the patient and an X-ray radiation dose profile therefore, are obtained, as described herein and/or otherwise. Examples of such images include the first scout image 402 (FIG. 4) and the second scout image 502 (FIG. 5). In another instance, other scout images for the patient are also obtained, including scout images corresponding to a same and/or different imaging examination, a same and/or different Z-axis reference origin, and/or with overlapping, contiguous and/or non-overlapping, and non-contiguous anatomical regions.

At 2404, a first mask image is generated for the first scout image and a second mask image is generated for the second scout image, as described herein and/or otherwise. Examples of such images include the first mask image 1202 (FIG. 12) for the first scout image 402 and the second mask image 1302 (FIG. 13) for the second scout image 502.

At 2406, shoulder and crotch markers are added to the first mask image and top of head and shoulder markers are added to the second mask image, as described herein and/or otherwise. Example images include the first mask image with the shoulder markers 1426 and 1428 and the crotch marker 1622 (FIGS. 14, 15 and 16) and the second mask image 1302 with the top of the head marker 1714 and the shoulder markers 1818 and 1820 (FIGS. 17 and 18).

At 2408, a reference image of a human, pre-marked with based on a predetermined set of anatomical regions and including top of the head, shoulder and crotch markers, is obtained, as described herein and/or otherwise. An example includes the human reference image 1902 (FIG. 19), which includes the head region 804, the neck and shoulder region 806, the chest region 808, the abdomen region 810, the pelvic region 812, and the legs region 814, and the top of the head marker 1904, the shoulder markers 1906 and 1908, and the crotch marker 1910.

At 2410, the first mask image and the second mask image are aligned with the human reference image based on the markers, as described herein and/or otherwise. An example is shown in FIG. 20, which shows the shoulder markers 1426 and 1428 of the first mask image 1202 aligned with the shoulder markers 1906 and 1908 of the human reference image 1902 and the crotch marker 1622 of the first mask image 1202 aligned with the crotch marker 1910 of the human reference image 1902, and the top of the head markers 1714 of the second mask image 1302 aligned with the top of the head markers 1904 of the human reference image 1902 and the shoulder markers 1818 and 1820 of the second mask image 1302 aligned with the shoulder markers 1906 and 1908 of the human reference image 1902.

At 2412, the anatomical regions of the reference image are mapped onto the first mask image and the second mask image, as described herein and/or otherwise. For example, the head region 804, the neck and shoulder region 806, the chest region 808, the abdomen region 810, the pelvic region 812 and the legs region 814 are mapped onto the first mask image 1202 and the head region 804 and the neck and shoulder region 806 are mapped onto the second mask image 1302.

At 2414, the anatomical regions are applied to be able to correctly sum the doses, as described herein and/or otherwise. As discussed above, since the first mask image 1202 and the first scout image 402 are a same size, and the second mask image 1302 and the second scout image 502 are a same size, the anatomical regions map from the first mask image 1202 to the first scout image 402 and from the second mask image 1302 map to the second scout image 502.

At 2416, the first scout image and the second scout image are combined with the human reference image, as described herein and/or otherwise. An example is shown in FIG. 21, which graphically depicts the human reference image 1902 with the first scout image 402 and the second scout image 502 superimposed or overlaid thereover and matched to the anatomical regions (i.e., the head region 804, the neck/shoulder region 806, the chest region 808, the abdomen region 810, the pelvic region 812, and/or the legs region 814).

At 2418, the X-ray dose of the first scout image and the X-ray dose of the second scout image projected onto the reference image are summed, generating a cumulative X-ray radiation dose profile over time that includes a cumulative X-ray radiation dose map, as described herein and/or otherwise. An example is graphically illustrated FIG. 10, the human reference (but with the human reference image 802 instead of the human reference image 1902) with the cumulative X-ray radiation dose map 1006 for the first scout images 402 and the second scout image 502.

At 2420, the cumulative X-ray radiation dose map (with or without other information) is displayed to a user and/or otherwise utilized. An example visual presentation is graphically illustrated FIG. 10, the human reference (but with the human reference image 802 instead of the human reference image 1902) with the cumulative X-ray radiation dose map 1006 for the first scout images 402 and the second scout image 502.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

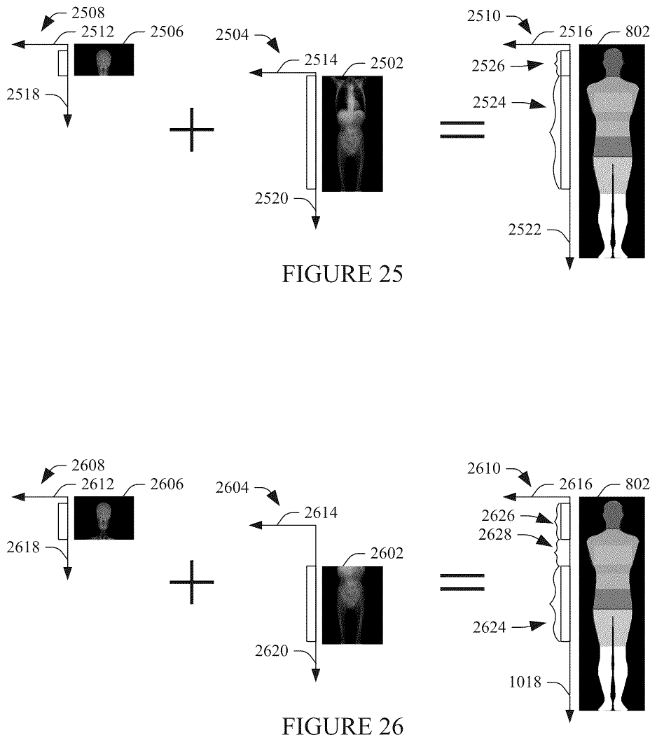

As discussed above, the approach described herein determines a cumulative patient X-ray radiation dose profile from scout images with overlapping, contiguous and/or non-overlapping, and non-contiguous anatomical regions. For sake of brevity and explanatory purposes, the above example include scout images with overlapping anatomical regions. FIGS. 25 and 26 respectively illustrate examples for determining a cumulative X-ray radiation dose profile with scout images having contiguous anatomical regions (FIG. 25) and a gap between anatomical regions (FIG. 26).

FIG. 25 graphically depicts a first scout image 2502 along with a corresponding dose plot 2504, a second scout image 2506 along with a corresponding dose plot 1004, and the human reference image 802 along with a cumulative dose plot 2510, which includes a summation of the plots 2502 and 2504. The plots 2502, 2504 and 2510 respectively include first axes 2512, 2514 and 2516 that represent dose and second axes 2518, 2520 and 2522 that represent a longitudinal axis of the patient. The cumulative dose plot 2516 includes a region 2524 that represents the dose plot 2504 corresponding to the first scout image 2502 and a region 2526 that represents the dose plot 2508 corresponding to the second scout image 2506. Similar to FIG. 10, the dose values in the plots 2504 and 2508 are the same in this example for explanatory purposes, and can be different, and the dose of each of the dose plots 2504 and 2508 may correspond to one or more volume scans.

FIG. 26 graphically depicts a first scout image 2602 along with a corresponding dose plot 2604, a second scout image 2606 along with a corresponding dose plot 1004, and the human reference image 802 along with a cumulative dose plot 2610, which includes a summation of the plots 2602 and 2604. The plots 2602, 2604 and 2610 respectively include first axes 2612, 2614 and 2616 that represent dose and second axes 2618, 2620 and 2622 that represent a longitudinal axis of the patient. The cumulative dose plot 2616 includes a region 2624 that represents the dose plot 2604 corresponding to the first scout image 2602, a region 2626 that represents the dose plot 2608 corresponding to the second scout image 2606, and a region 2628 that represents a gap between anatomical regions in the scout images 2602 and 2606. Similar to FIG. 10, the dose values in the plots 2604 and 2608 are the same in this example for explanatory purposes, and can be different, and the dose of each of the dose plots 2604 and 2608 may correspond to one or more volume scans.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A computer-implemented method, comprising:
receiving at least a first scout image with a first dose profile and a second scout image with a second dose profile for a patient,
wherein the first scout image includes a first reference origin, the second scout image includes a second reference origin;
receiving a human reference image that is pre-marked with a set of anatomical regions;
mapping anatomical regions between the first scout image and the human reference image and the second scout image and the human reference image;
summing the first dose profile and the second dose profile based on the mapped anatomical regions between the first and second scout images and the human reference image, independent of the first and second reference origins; and
generating a cumulative dose profile for the patient based on the summed first and second dose profiles.

2. The computer-implemented method of claim 1, further comprising:
detecting anatomical regions in each of the first scout image and the second scout image based on the set of anatomical regions;
aligning anatomical regions of the first scout image anatomical regions of the human reference image and anatomical regions of the second scout image anatomical regions of the human reference image; and
projecting the first dose profile to human reference image based on the aligned anatomical regions of the first scout image and the second dose profile to human reference image based on the aligned anatomical regions of the second scout image.

3. The computer-implemented method of claim 2, further comprising:
aligning the first and second scout images with the human reference image based on a first anatomical region that is common to the first and second scout images and the human reference image.

4. The computer-implemented method of claim 3, further comprising:
further aligning the first scout image with the human reference image based on a second anatomical region that is common to the first scout image and the human reference image; and
further aligning the second scout image with the human reference image based on a third anatomical region that is common to the second scout image and the human reference image.

5. The computer-implemented method of claim 4, wherein the aligning includes resizing at least one of the first scout image and the second scout image to match dimensions of the human reference image.

6. The computer-implemented method of claim 4, wherein the human reference image is further pre-marked with a set of anatomical markers, and further comprising:
generating a first mask image for the first scout image;
generating a second mask image for the second scout image;
positioning at least first and second anatomical marker of the set of anatomical markers at corresponding first and second locations in the first mask image, wherein the first and second locations are different locations;
positioning at least third and fourth anatomical marker of the set of anatomical markers at corresponding third and fourth locations in the second mask image, wherein the third and fourth locations are different locations;
aligning the first mask images with the human reference image based on the first and second anatomical markers;
aligning the second mask images with the human reference image based on the third and fourth anatomical markers; and
summing the first and second dose profiles based on the first and second mask images aligned with the human reference image.

7. The computer-implemented method of claim 6, further comprising:
combining the first and second scout images and the human reference image; and
summing the first and second dose profiles of the first and second scout images based on the combined first scout image, second scout image, and human reference image.

8. The computer-implemented method of claim 7, wherein positioning the first, second, third and fourth anatomical markers include:
identifying pixel patterns in at least one of the first and second mask images that correspond to boundaries between regions outside of the patient and anatomical regions of the patient adjacent to the regions outside of the patient; and
identifying pixel patterns in at least one of the first and second mask images that correspond to boundaries between adjacent anatomical regions of the patient.

9. The computer-implemented method of claim 1, wherein the human reference image is further pre-marked with a set of anatomical markers, and further comprising:
generating a first mask image for the first scout image;
generating a second mask image for the second scout image;
positioning at least first and second anatomical marker of the set of anatomical markers at corresponding first and second locations in the first mask image, wherein the first and second locations are different locations;
positioning at least third and fourth anatomical marker of the set of anatomical markers at corresponding third and fourth locations in the second mask image, wherein the third and fourth locations are different locations;
aligning the first mask image with the human reference image based on the first and second anatomical markers;
aligning the second mask images with the human reference image based on the third and fourth anatomical markers; and
summing the first and second dose profiles based on the first and second mask images aligned with the human reference image.

10. The computer-implemented method of claim 9, further comprising:
combining the first and second scout images and the human reference image; and
summing the first and second dose profiles of the first and second scout images based on the combined first scout image, second scout image, and human reference image.

11. The computer-implemented method of claim 10, wherein positioning the first, second, third and fourth anatomical markers include:
identifying pixel patterns in at least one of the first and second mask images that correspond to boundaries between regions outside of the patient and anatomical regions of the patient adjacent to the regions outside of the patient; and
identifying pixel patterns in at least one of the first and second mask images that correspond to boundaries between adjacent anatomical regions of the patient.

12. The computer-implemented method of claim 1, further comprising:
displaying at least the human reference image and the summed first and second dose profiles as a cumulative dose map.

13. A computer readable medium encoded with computer executable instructions, which, when executed by a processor, causes the processor to:
receive at least a first scout image with a first dose profile and a second scout image with a second dose profile for a patient,
wherein the first scout image includes a first reference origin, the second scout image includes a second reference origin;
receive a human reference image that is pre-marked with a set of anatomical regions;
map anatomical regions between the first scout image and the human reference image and the second scout image and the human reference image;
sum the first dose profile and the second dose profile based on the mapped anatomical regions between the first and second scout images and the human reference image, independent of the first and second reference origins; and
generate a cumulative dose profile for the patient based on the summed first and second dose profiles.

14. The computer readable medium of claim 13, wherein the computer executable instructions further cause the processor to:
detect anatomical regions in each of the first scout image and the second scout image based on the set of anatomical regions;
align anatomical regions of the first scout image anatomical regions of the human reference image and anatomical regions of the second scout image anatomical regions of the human reference image; and
project the first dose profile to human reference image based on the aligned anatomical regions of the first scout image and the second dose profile to human reference image based on the aligned anatomical regions of the second scout image.

15. The computer readable medium of claim 14, wherein the computer executable instructions further cause the processor to:
align the first and second scout images with the human reference image based on a first anatomical region that is common to the first and second scout images and the human reference image.

16. The computer readable medium of claim 15, wherein the computer executable instructions further cause the processor to:
further align the first scout image with the human reference image based on a second anatomical region that is common to the first scout image and the human reference image; and
further align the second scout image with the human reference image based on a third anatomical region that is common to the second scout image and the human reference image.

17. The computer readable medium of claim 13, wherein the computer executable instructions further cause the processor to:
generate a first mask image for the first scout image;
generate a second mask image for the second scout image;
position at least first and second anatomical marker of the set of anatomical markers at corresponding first and second locations in the first mask image, wherein the first and second locations are different locations;
position at least third and fourth anatomical marker of the set of anatomical markers at corresponding third and fourth locations in the second mask image, wherein the third and fourth locations are different locations;
align the first mask images with the human reference image based on the first and second anatomical markers;
align the second mask images with the human reference image based on the third and fourth anatomical markers; and
sum the first and second dose profiles based on the first and second mask images aligned with the human reference image.

18. The computer readable medium of claim 14, wherein the computer executable instructions further cause the processor to:
combine the first and second scout images and the human reference image; and
sum the first and second dose profiles of the first and second scout images based on the combined first scout image, second scout image, and human reference image.

19. The computer readable medium of claim 15, wherein the computer executable instructions further cause the processor to, at least one of:
identify pixel patterns in at least one of the first and second mask images that correspond to boundaries between regions outside of the patient and anatomical regions of the patient adjacent to the regions outside of the patient; and
identify pixel patterns in at least one of the first and second mask images that correspond to boundaries between adjacent anatomical regions of the patient.

20. A system, comprising:
a memory including a cumulative X-ray radiation dose generating module;
at least one processor configured to execute instructions of the cumulative X-ray radiation dose generating module,
wherein the instructions cause the processor to:
receive at least a first scout image with a first dose profile and a second scout image with a second dose profile for a patient,
wherein the first scout image includes a first reference origin, and the second scout image includes a second reference origin;
receive a human reference image that is pre-marked with a set of anatomical regions;
map anatomical regions between the first scout image and the human reference image and the second scout image and the human reference image;
sum the first dose profile and the second dose profile based on the mapped anatomical regions between the first and second scout images and the human reference image, independent of the first and second reference origins; and
generate a cumulative dose profile for the patient based on the summed first and second dose profiles.
